(19) United States
(12) Patent Application Publication
(10) Pub. No.: US 2006/0182657 A1
(43) Pub. Date: Aug. 17, 2006

(54) Devices and methods for handling and processing punches

(75) Inventors: Navin Deepal Pathirana, Florham Park, NJ (US); Stevan Paul Tortorella, Wells, ME (US)

Correspondence Address: EDWARDS & ANGELL, LLP, P.O. BOX 55874, BOSTON, MA, 02205, US

(73) Assignee: Whatman, Inc., Florham Park, NJ (US)

(21) Appl. No.: 11/352,707

(22) Filed: Feb. 10, 2006

Related U.S. Application Data
(60) Provisional application No. 60/652,234, filed on Feb. 11, 2005.

(51) Int. Cl. G01N 33/00

(52) U.S. Cl. 422/63

(57) ABSTRACT

The present invention provides devices and methods for handling and processing a filter or other matrix punch comprising a sample of interest in order to prevent loss of the punch and to improve ease of handling. In one aspect, the present invention provides a device comprising a first element, which holds a punch, coupled to a second element, which contributes to holding the punch in the first element, with reservoirs above and below the punch. The devices and methods can be used manually in single-channel and multi-channel formats or can be used in an automated processor, such as a robotic processor. Kits are also provided.

This application claims priority from U.S. Provisional Patent Application No. 60/652,234 entitled “DEVICES AND METHODS FOR HANDLING AND PROCESSING PUNCHES” filed Feb. 11, 2005, the entire content of which is incorporated herein by reference.

### FIELD OF THE INVENTION

The present invention provides devices and methods for handling and processing a filter or other matrix punch comprising a sample of interest in order to prevent loss of the punch and to improve ease of handling. The devices and methods can be used manually in single-channel and multi-channel formats or can be used in an automated processor, such as a robotic processor. Kits are also provided.

### BACKGROUND OF THE INVENTION

Methods of archiving nucleic acids or proteins, such as by storing a sample on a filter, card, and other type of matrix, are well known in the art. Examples include those described in WO 90/03959 (PCT/AU89/00430; filed 3 Oct. 1989), WO 96/39813 (PCT/AU96/00344; filed 7 Jun. 1996), WO 00/21973 (PCT/GB99/03337; filed 8 Oct. 1999), WO 01/501601 (PCT/US01/00640; filed 10 Jan. 2001), WO 03/020924 (PCT/GB02/04048; filed 5 Sep. 2002), PCT/US01/25709 (filed 17 Aug. 2001), PCT/US03/31483 (filed 3 Oct. 2003), U.S. Pat. No. 5,496,562 (granted 5 Mar. 1996), U.S. Pat. No. 5,756,126 (granted 26 May 1998), U.S. Pat. No. 5,939,259 (granted 17 Aug. 1999), U.S. Pat. No. 5,972,386 (granted 28 Oct. 1999), U.S. Pat. No. 6,168,922 (granted 2 Jan. 2001), U.S. Pat. No. 6,291,179 (granted 18 Sep. 2001), U.S. Pat. No. 6,645,717 (granted 11 Nov. 2003), U.S. Pat. No. 6,670,128 (granted 30 Dec. 2003), U.S. Ser. No. 09/724,060 (filed 28 Nov. 2000), U.S. Ser. No. 09/993,736 (filed 14 Nov. 2001), U.S. Ser. No. 10/326,216 (filed 20 Dec. 2002), European Patent 1119576 (11 Jun. 2003), European Patent 0849992 (25 Aug. 2004), and European Patent Application 04076551.3 (filed 20 May 2004).

Matrices may be made of a wide range of materials, including cellulose, glass and other silica-based substances, and plastic materials, and optionally may be treated, such as with a chemical composition. In some instances, the matrix may be able to be stored at room temperature for many months while preserving the sample. The sample may be bound or sorbed to the matrix, either directly or indirectly, by physical, chemical, or other interactions. In time, however, the sample will need to be analyzed. Typically, a punch or micro-punch is made in a part of the matrix containing the sample for analysis, while the remaining portion of the sample on the matrix continues to be stored. For some analyses, a separate elution or release may not be necessary (see, e.g., U.S. Pat. No. 6,750,059 (granted 15 Jun. 2004), U.S. Pat. No. 6,746,841 (granted 8 Jun. 2004), U.S. Ser. No. 10/298,255 (filed 15 Nov. 2002)), but for others, isolation of the nucleic acid or protein may be desirable or essential. Isolation methods include isolation using elution (e.g., by heat, by change in pH or salt concentration); enzymatic methods; photolysis; a combination of these methods; or by other means (see, e.g., WO 01/501601 (PCT/US01/00640; filed 10 Jan. 2001), PCT/US01/25709 (filed 17 Aug. 2001), PCT/US02/36483 (filed 13 Nov. 2002), WO 03/020924 (PCT/GB02/04048 filed 5 Sep. 2002), U.S. Pat. No. 6,645,717 (granted 11 Nov. 2003), U.S. Pat. No. 6,670,128 (granted 30 Dec. 2003)). Whether or not the sample is isolated, however, the punch will require processing.

For example, currently a general method for processing punches (e.g., FTA® punches) where elution is required is as follows:
a) Take a punch from the relevant sample (e.g., FTA® CLONESAVER® card);
b) Place punch in a tube (e.g., micro-centrifuge tube);
c) Add wash buffer and rinse punch;
d) Remove wash buffer (repeat steps (c) & (d) as necessary);
e) Add elution buffer and incubate (at room or higher temperature);
f) Remove eluate and discard punch.

The eluate isolated in step (f) will contain the material of interest. The material may be a nucleic acid (e.g., genomic DNA, plasmid DNA, mitochondrial DNA, total RNA, siRNA, mRNA, etc.), protein(s) or any other material of interest (e.g., peptide, oligonucleotide, etc.).

The disadvantage of this method of handling the punch includes loss of punch during liquid removal stages. This is especially true in automated systems where a wet punch may stick to the pipet tip, particularly without being detected. Small punches can also be dislodged from the tube due to static build-up.

In the past there have been attempts to address the issue of handling punches. Alternative approaches have also been attempted. For example, Millipore has a system called ZIPTIP® for purifying biomolecules based on a pipet tip. ZIPTIPs® have chromatography media (silica based) (e.g. C₁₈, C₄ or chelating resin) immobilised within a pipet tip by use of a polymeric scaffold. The advantage of the ZIPTIP® is that handling and processing using the tip is made easy by the fact that tip attaches to a 10 μl pipettor. The sample is aspirated and dispensed a few times to bind the substance of interest. The tip is then washed with a wash solution to remove any impurities and finally, the substance of interest is eluted from the tip using a suitable solvent. A single tip can be processed using a single channel pipettor. Eight tips can be processed at the same time by use of an eight channel pipettor. Automated liquid handling systems can also be used. The use of ZIPTIPs® is limited, however, to chromatography media supplied by the manufacturer. The user cannot add any solids to the tip. This tip is not intended for use with punches but it uses a pipettor to process the tips.

Accordingly, it would be desirable to have a device and methods to facilitate the handling and processing of punches.

### SUMMARY OF THE INVENTION

In one aspect, the present invention provides a device for processing a punch from a matrix comprising a sample of interest, wherein the device comprises:
a. a first element comprising a dispensing tip comprising:
   i. a hollow internal shaft having an external opening; and
   ii. a first housing assembly structured and arranged to define a first inner reservoir communicating with the internal shaft of the dispensing tip, wherein the dimensions of the first inner reservoir are selected such that a punch inserted therein divides the first inner reservoir into two chambers, wherein:
      the first chamber communicates with the internal shaft of the dispensing tip; and
      the second chamber comprises a coupling opening; and
b. a second element comprising a second housing assembly structured and arranged to define:
   i. a hollow interior;
   ii. a coupling portion for engaging the second element to the first element; and
   iii. a second coupling opening for communication through the first coupling opening of the first element, between the hollow interior of the second element and the first inner reservoir, wherein the dimensions of the second housing assembly are selected such that, when a punch is positioned in the first inner reservoir of the first element, the rim defining the second coupling opening contributes to maintain the position of the punch within the first inner reservoir and forms a second inner reservoir within the hollow interior of the second element.

In another aspect, the present invention provides a kit for processing a punch from a matrix comprising a sample of interest, wherein the kit comprises:

a. the device; and

b. a processing reagent.

In yet another aspect, the present invention provides a method of processing a punch from a matrix comprising a sample of interest, wherein the method comprises:
a. punching a matrix to yield a punch comprising a sample of interest;
b. providing the device of any one of the preceding claims;
c. inserting the matrix into the first housing assembly of the first element of the device;
d. coupling the first element to the second element;
e. processing the punch with a processing reagent.

### BRIEF DESCRIPTION OF THE DRAWINGS

FIG. 1 is a schematic of the lower section of an embodiment of the present invention.

FIG. 2 is a schematic of the upper section of an embodiment of the present invention.

FIG. 3 is a schematic of the lower and upper sections of FIGS. 1 and 2 in combination with reference to a pipettor tip.

FIG. 4 is a schematic of the combination of FIG. 3 with reference to a buffer station.

FIGS. 5A and 5B are schematics of two embodiments of the present invention using a syringe.

### DETAILED DESCRIPTION OF THE INVENTION

In one aspect, the present invention provides a device for processing a punch from a matrix comprising a sample of interest, wherein the device comprises:
a. a first element comprising a dispensing tip comprising:
   i. a hollow internal shaft having an external opening; and
   ii. a first housing assembly structured and arranged to define a first inner reservoir communicating with the internal shaft of the dispensing tip, wherein the dimensions of the first inner reservoir are selected such that a punch inserted therein divides the first inner reservoir into two chambers, wherein:
      the first chamber communicates with the internal shaft of the dispensing tip; and
      the second chamber comprises a coupling opening; and
b. a second element comprising a second housing assembly structured and arranged to define:
   i. a hollow interior;
   ii. a coupling portion for engaging the second element to the first element; and
   iii. a second coupling opening for communication through the first coupling opening of the first element, between the hollow interior of the second element and the first inner reservoir, wherein the dimensions of the second housing assembly are selected such that, when a punch is positioned in the first inner reservoir of the first element, the rim defining the second coupling opening contributes to maintain the position of the punch within the first inner reservoir and forms a second inner reservoir within the hollow interior of the second element.

In one embodiment, the first inner reservoir further comprises a punch support, wherein the dimensions of the punch support are selected such that most of the surface area of the punch placed on the punch support is accessible to a fluid in the first chamber when a punch is placed on the punch support and the first chamber is filled with the fluid. Preferably, the punch support comprises an O-ring or a ribbed support with a series of channels for fluid flow.

In another embodiment, the coupling portion engages the second element to the first element by a pressure lock or by mechanical locking mechanism. Preferably, the mechanical locking mechanism comprises a snap fitting or a Luer lock.

In another embodiment, the first inner reservoir is contiguous with the hollow inner shaft within the first housing assembly.

In yet another embodiment, the dispensing tip of the first element comprises a micro-dispensing pipet tip. Preferably, the micro-dispensing pipet tip comprises a capillary micro-dispensing pipet tip.

In another embodiment, the second element comprises a micro-dispensing pipet tip.

In another embodiment, the device is adapted for use with a micro-dispensing pipet selected from the group consisting of:

a. a single-channel micro-dispensing pipet;

b. a multi-channel micro-dispensing pipet; and

c. an automated micro-dispensing pipet machine or robot.

Alternatively, the second element comprises a syringe selected from the group consisting of:

a. a manual syringe; and

b. a robotic syringe.

Preferably, the first element is adapted to be fitted onto a syringe, wherein the hollow internal shaft is housed in a syringe needle or the first housing assembly is adapted to be fitted between a syringe and a syringe needle.

In yet another embodiment, the device further comprises:

c. a heating element.

In another aspect, the present invention provides a kit for processing a punch from a matrix comprising a sample of interest, wherein the kit comprises:

a. the device; and

b. a processing reagent.

In one embodiment, the processing reagent comprises an elution buffer. Preferably, the elution buffer is selected from the group consisting of NaOH, sodium acetate, 10 mM 2-[N-morpholino]-ethanesulfonic acid (MES), 10 mM 3-[cyclohexylamino]-1-propanesulfonic acid (CAPS), TE, TE⁻¹, sodium dodecyl sulfate (SDS), an aqueous solution of sorbitan mono-9octadecenoate poly(oxy-1,1-ethanedlyl), lauryl dodecyl sulfate (LDS), or t-octylphenoxypolyethoxyethanol, 10 mM Tris, phosphate buffered saline (PBS), and water.

In another embodiment, the processing reagent comprises an indicator.

In another embodiment, the processing reagent comprises an enzyme or a photolytic agent.

In still another embodiment, the kit further comprises:

c. a heating element.

In yet another embodiment, the kit further comprises:

c. a punch comprising a sample of interest.

Preferably, the sample of interest comprises:

a. a nucleic acid; or

b. a protein or a peptide.

More preferably, wherein the sample of interest comprises a nucleic acid selected from the group consisting of genomic DNA, plasmid DNA, mitochrondrial DNA, cDNA, an oligonucleotide, viral DNA or RNA, BAC, mRNA, rRNA, tRNA, siRNA, and total RNA.

In yet another aspect, the present invention provides a method of processing a punch from a matrix comprising a sample of interest, wherein the method comprises:
a. punching a matrix to yield a punch comprising a sample of interest;
b. providing the device of any one of the preceding claims;
c. inserting the matrix into the first housing assembly of the first element of the device;
d. coupling the first element to the second element;
e. processing the punch with a processing reagent.

In one embodiment, the processing step comprises:
i. drawing the processing reagent into the first element so that the processing reagent contacts the punch;
ii. removing the processing reagent from the first element.

In another embodiment, the processing step comprises:
i. drawing the processing reagent into the first element so that the processing reagent contacts the punch and moves past it into the second chamber;
ii. pushing the processing reagent through the punch into the first chamber;
iii. removing the processing reagent from the first element.

Preferably, step i. further comprises incubation of the punch.

In a preferred embodiment, the coupling step comprises locking by pressure or by a mechanical lock.

In another embodiment, the processing reagent comprises an elution buffer. Preferably, the elution buffer is selected from the group consisting of NaOH, sodium acetate, 10 mM 2-[N-morpholino]-ethanesulfonic acid (MES), 10 mM 3-[cyclohexylamino]-1-propanesulfonic acid (CAPS), TE, TE⁻¹, sodium dodecyl sulfate (SDS), an aqueous solution of sorbitan mono-9octadecenoate poly(oxy-1,1-ethanedlyl), lauryl dodecyl sulfate (LDS), or t-octylphenoxypolyethoxyethanol, 10 mM Tris, phosphate buffered saline (PBS), and water.

Preferably, the elution buffer is heated to a temperature of between 40° C. to 125° C., wherein:
a. the elution buffer is heated prior to contact with the punch; or
b. the elution buffer is heated during contact with the punch in the first housing assembly.

More preferably, the elution buffer is heated to a temperature of between 65° C. and 95° C.

In another embodiment, the processing reagent comprises an indicator.

In yet another embodiment, the processing reagent comprises an enzyme or a photolytic agent.

In another embodiment, the sample of interest comprises a nucleic acid. Preferably, the nucleic acid is selected from the group consisting of genomic DNA, plasmid DNA, mitochrondrial DNA, cDNA, an oligonucleotide, viral DNA or RNA, BAC, mRNA, rRNA, tRNA, siRNA, and total RNA.

In still another embodiment, the sample of interest comprises a protein or a peptide.

In yet another embodiment, the matrix comprises:

a. a cellulose-based matrix;

b. a silica-based matrix; or

c. a plastics-based matrix.

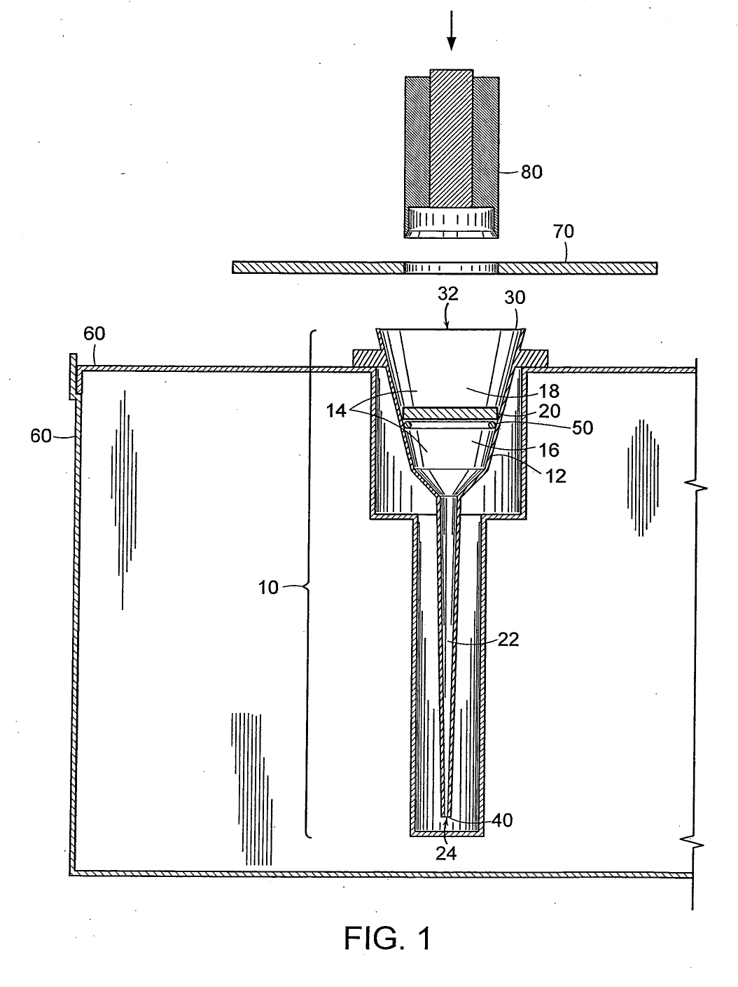

A device is provided which may, in one aspect, be used in the extraction of samples such as blood according to the method described above. Such a device is depicted in FIGS. 1-4.

In one embodiment, the device consists essentially of two elements or sections, shown supported by a holder in FIGS. 1-3. The first element (10), here the lower section, is shown in FIG. 1. In this embodiment, the first element (10) is depicted as a dispensing pipet tip having a hollow internal shaft (22) with an external opening (24) and a first housing assembly (12). The first housing assembly (12) is designed to house the first inner reservoir (14).

The punch (20) (such as a punch from an FTA® CLONESAVER® archival card) is placed in this section of the device by punch and place equipment. In this embodiment, the punch rests on a punch support (50), such as an O-ring.

In one alternative, the punch support comprises a ribbed support with a series of channels for fluid flow. An example of the latter is essentially a disc, which has ribs (in the shape of concentric rings or a star). The filter sits on top of the ribs creating a gap underneath the filter. Liquid flows in to these gaps and is channeled out from a hole in the center of the disc. An analogous device is used in syringe filters (Whatman EASYDISC™, GD/X™, GD/XP™), but is adapted as a support for the present invention.

In another alternative, the punch may simply rest on the inside wall of the hollow internal shaft (22), due to a decreased radius. Once the punch (20) is placed in the first inner reservoir (14), it divides the first inner reservoir (14) into a first chamber (16), which communicates with the hollow internal shaft (22), and a second chamber (18), which has a coupling opening (32). In this embodiment, the coupling opening (32) is defined by an edge or rim (30).

In the embodiment depicted in FIG. 1, the distance between the top edge (30) of the lower section (10) and the punch support (50) is kept shallow to allow easy placement of the punch (20). In this embodiment, the tip (40) of the lower section (10) is shown as a capillary tip, such as a gel loading tip, which minimizes the hold up volume of the tip.

The lower the hold up volume of the tip, the greater will be the recovery of the material of interest. This is especially true when the elution volume is small (e.g., 25 μl). In these instances, a hold up volume of, e.g., 10 μl is a significant percentage of the elution volume.

In FIG. 1, the lower section (10) is shown held in a holder (60). In one embodiment, holder is, for example, of a 96 well type (i.e., it holds 96 tips in the same footprint as a standard 96 well plate). Once the punches are placed on the lower section, these parts (still in the holder) are used manually (e.g., in a single-channel or multi-channel pipettor) or transferred to a liquid handling robot. In embodiments using a robot, the robot may also be loaded with the upper section of the device.

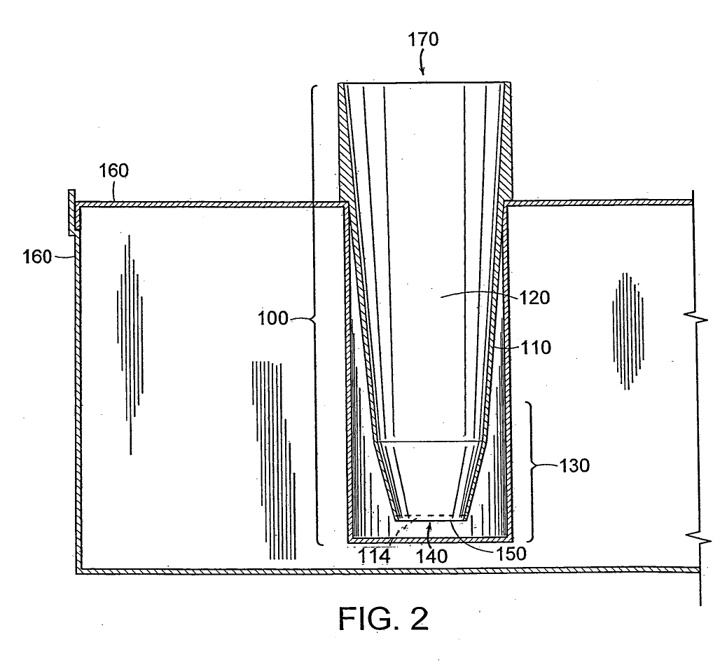

The second element, or upper section (100) of this embodiment of the device is depicted in FIG. 2, here depicted in a holder (160). In FIG. 2, the upper section (100) is depicted as a dispensing pipet tip, which has a second housing assembly (110) defining a hollow interior (120). In this embodiment, this second dispensing pipet tip is larger than the first tip used as the lower section (10).

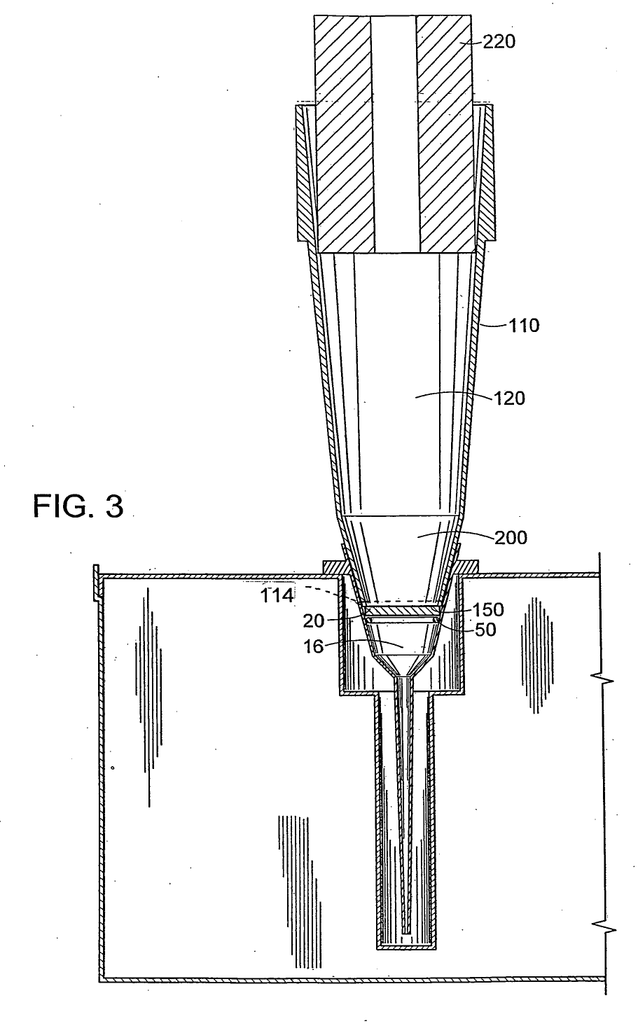

The second housing assembly (110) is structured and arranged to define a coupling portion (130) for communication through the first coupling opening (32) of the lower section (10), between the hollow interior (120) of the upper section (100) and the second chamber (18) of the first inner reservoir (14). In this embodiment, the coupling portion (130) ends with a rim or edge (150), which defines the second coupling opening (140), which communicates with the lower section (10) (see FIGS. 2 and 3). Also, a punch retention portion, generally indicated at (114), may be located within the opening (140) at, or substantially adjacent to, the end of the upper section (100) that includes the rim or edge (150). While various alternative configurations may be adopted as the punch retention portion, in preferred embodiments the punch retention portion (114) is a flexible element in the shape of a bar or a cross that extends across the opening (140) in the plane containing, or a plane substantially parallel the to plane containing, the rim or edge (150). Further, the punch retention portion (114) is sized relative to the opening (140) such that it does not significantly impede a flow of liquid through the opening (140), but yet acts to retain the punch against the tendency for it to be displaced and/or to be drawn into the hollow interior (120) of the upper section (100) during the punch processing steps described in detail below.

In one embodiment, this part is designed to be picked up directly or indirectly by a user or by a standard liquid handling robot and be pushed into the lower section in a manner similar to that shown in FIG. 3.

FIG. 3 depicts the two elements, or sections, joined. As shown in FIG. 2, the dimensions of the second housing assembly (110) are selected such that, when a punch (20) is positioned in the first inner reservoir (14) of the lower section (10), the rim or edge (150) defining the second coupling opening (140) contributes to maintain the position of the punch (20) within the first inner reservoir (14) and forms a second inner reservoir (200) housed within the hollow interior (120) of the second housing assembly (110), which is interior to the second chamber (18) of the lower section (10).

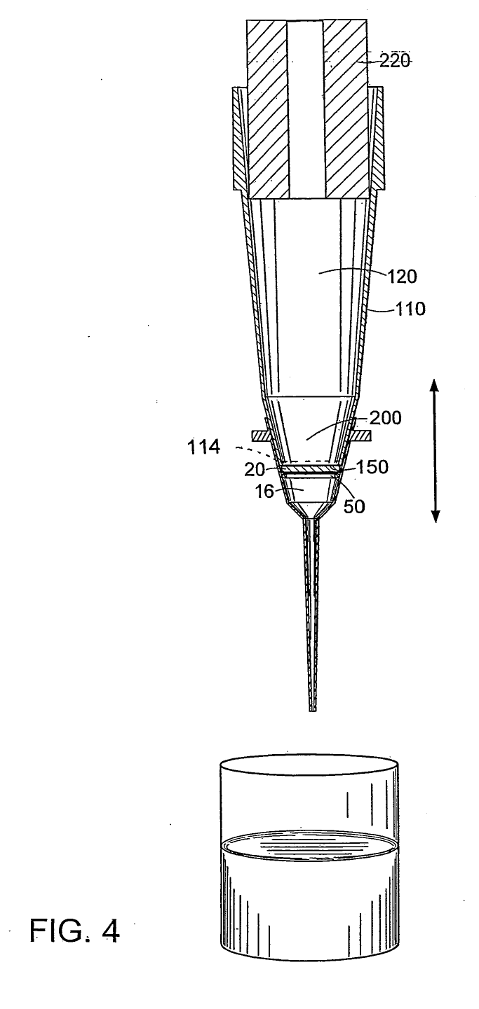

Preferably, once the two parts are pushed together, the two sections lock together (e.g., by pressure or by a locking mechanism or element, such as a snap fitting or a Luer lock), and the device can be moved as a single piece. The upper section performs three functions: i) holds the punch in place during processing steps, ii) acts as a reservoir for liquid when liquid is aspirated through the punch during the processing steps, and iii) enables the whole device to be handled by a user or liquid pipetting robot (see FIG. 4), such as using a pipettor (220), which can communicate force through an opening (170) in the upper section (100) into the hollow interior (120) through the second inner reservoir (200).

Once the upper and lower sections are locked together, the user or liquid handling robot picks up the device and takes the device to a buffer station, as depicted in FIG. 4. (Robots can handle multiple devices simultaneously. Alternatively, a non-robotic user can handle multiple devices using a multi-channel pipettor.) The punch (20) can be washed (if desired) by aspirating liquid (e.g., hot or cold aqueous buffers, alcohols, organic solvents, etc.) into the device and pulsing the liquid up and down through the punch or by aspirating and dispensing liquid from the device. After washing, the elution buffer is aspirated in to the device and incubated. Buffer is taken up in to the tip and moved up and down through the punch to wash and elute. If heating is necessary at the elution stage the whole tip is placed in a heating block. After incubation, the liquid from the device is dispensed in to a collection vessel, and the device is discarded. The design makes possible the use of relatively large (e.g., 200 μl) buffer volumes during washing stages and the use of small volumes during the elution stage.

An alternative to the customer punching and placing the punch is for the manufacturer of the device to provide a device pre-loaded with a punch (e.g., provide a lower section pre-loaded with a FTA® punch). The customer then loads the lower-sections in to liquid handling robot and spots the sample of interest on to the punch using the robot. The upper sections are then pushed in to place (i.e., assemble the upper and lower sections together). The spot is allowed to dry and the devices are then placed in storage until required. When needed, the devices are processed as described above to obtain the material of interest (e.g., DNA, RNA, protein, etc.).

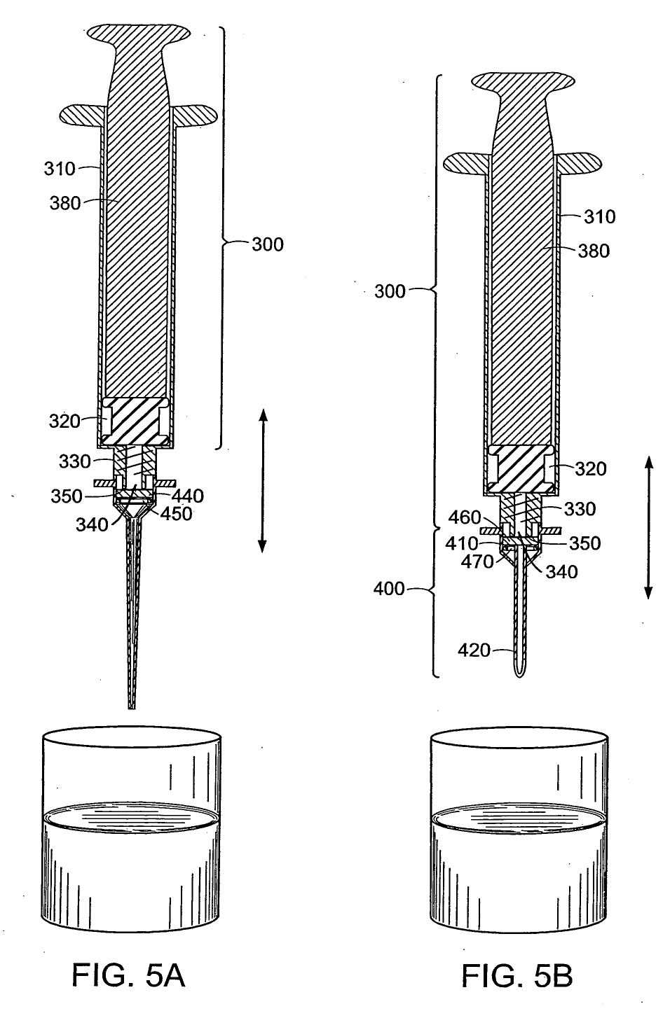

As an alternative to the device of FIGS. 1-4, a modified syringe device may be used, fitted either to a first element similar to the lower section described above (e.g., as shown in FIG. 5A), or to a modified needle. The size of the needle is selected to prevent loss of the punch. Alternatively, the size of the punch is selected to prevent its loss when using a specifically sized needle. In addition, the length and bore of the needle are selected with reference to the sample of interest being isolated. For example, a wide bore needle is used when the sample of interest comprises long strands of DNA, because shear forces in a narrow bore needle can break the strands.

In FIG. 5A, a first element (lower section), analogous to that of FIGS. 1-4, is shown attached to a syringe (300), the housing assembly (310) of which is structure and arranged to define the hollow interior (320) of the syringe (300). A coupling portion (330) of the syringe (300) couples the syringe (300) to the coupling opening of the first element so that the opening (340) of the syringe (300) communicates with the first inner reservoir. The rim or edge (350) of the opening contributes to maintaining the position of the punch (440), which is preferably positioned on a punch support (450). The two sections lock together (e.g., by pressure or by a locking mechanism or element, such as a snap fitting or a Luer lock). The syringe plunger (380) is used to draw liquid in and out.

In FIG. 5B, the first element (lower section) (400) has a housing assembly (410) connected to a syringe or other needle (420). The rim or edge (350) of the opening contributes to maintaining the position of the punch (460), which is preferably positioned on a punch support (470).

As an alternative to the devices shown in FIGS. 5A and 5B, the syringe needle is modified to have an extra-long connector region, which connects the upper end of the needle to the bottom end of the syringe. The connector region has a punch support onto which the punch is placed prior to attachment to the syringe.

As another alternative to the devices shown in FIGS. 5A and 5B, the connector region decreases in interior radius so that the punch rests on the interior wall, but with a space below it to maintain room for buffers or other liquids between the underside of the punch and the top end of the needle. The upper side of the punch is pressed down by the nozzle of the syringe when the syringe is attached. Alternatively, an upper O-ring is inserted on top of the punch prior to attachment to the syringe.

As yet another alternative to the device shown in FIG. 5B, an adaptor is provided and is placed between the syringe needle and the syringe. The adaptor is capable of fastening to the syringe nozzle at its upper end and to the connector region of the needle at its lower end, preferably using standard methods to allow commercially available syringes and needles to be used. The adaptor optionally has a punch support onto which the punch is placed prior to attachment to the syringe. The upper side of the punch is pressed down by the nozzle of the syringe when the syringe is attached. Alternatively, an upper O-ring is inserted on top of the punch prior to attachment to the syringe. In one embodiment, the adaptor has a punch already provided, either with or without a sample of interest.

Suitable materials include glass fiber or any silica-based or derived filters, cellulose-based filters, and plastic based filters, for example polyester and polypropylene based filters. Examples include those described in WO 90/03959 (PCT/AU89/00430; filed 3 Oct. 1989), WO 96/39813 (PCT/AU96/00344; filed 7 Jun. 1996), WO 00/21973 (PCT/GB99/03337; filed 8 Oct. 1999), WO 01/501601 (PCT/US01/00640; filed 10 Jan. 2001), WO 03/020924 (PCT/GB02/04048; filed 5 Sep. 2002), PCT/US01/25709 (filed 17 Aug. 2001), PCT/US03/31483 (filed 3 Oct. 2003), U.S. Pat. No. 5,496,562 (granted 5 Mar. 1996), U.S. Pat. No. 5,756,126 (26 May 1998), U.S. Pat. No. 5,939,259 (granted 17 Aug. 1999), U.S. Pat. No. 5,972,386 (granted 28 Oct. 1999), U.S. Pat. No. 6,168,922 (granted 2 Jan. 2001), U.S. Pat. No. 6,291,179 (granted 18 Sep. 2001), U.S. Pat. No. 6,645,717 (granted 11 Nov. 2003), U.S. Pat. No. 6,670,128 (granted 30 Dec. 2003), U.S. Ser. No. 09/724,060 (filed 28 Nov. 2000), U.S. Ser. No. 09/993,736 (filed 14 Nov. 2001), U.S. Ser. No. 10/326,216 (filed 20 Dec. 2002), European Patent 1119576 (11 Jun. 2003), European Patent 0849992 (25 Aug. 2004), and European Patent Application 04076551.3 (filed 20 May 2004), the disclosures of all of which are incorporated herein by reference.

If washing of a nucleic acid sample is desired, various washes can be performed in various types of buffers, including, but not limited to, hot or cold aqueous buffers, alcohols, and organic solvents. Preferably, the washing buffers can be selected from the group including Tris/EDTA; 70% ethanol; STET (0.1 M NaCl; 10 mM Tris/HCl, pH 8.0; 1 mM EDTA, pH 8.0; 5% Triton X-100); SSC (20×SSC=3 M NaCl; 0.3 M sodium citrate; pH 7.0 with NaOH); SSPE (20×SSPE=3 M NaCl; 0.2 M NaH₂PO₄—H₂O; 0.02 M EDTA; pH 7.4), and the like.

Elution buffers and protocols will depend on what is being eluted (e.g., plasmid DNA, genomic DNA, mRNA, protein, etc.) and which type of filter material is being used (e.g., cellulose-based, glass or silica-based, plastics-based, etc.).

It is preferred also that the filter composition and dimensions are selected so that the nucleic acid during elution is capable of being eluted at a pH of from pH 5 to 11 or preferably from pH 5.8 to 10. This is advantageous in the present method because elution of the product nucleic acid in a more highly alkaline medium potentially can degrade the product. Accordingly, one preferred pH for elution is from 7 to 9.

Eluting the nucleic acid, in other words releasing the nucleic acid from the filter, may be affected in several ways. The efficiency of elution may be improved by putting energy into the system during an incubation step to release the nucleic acid prior to elution. This may be in the form of physical energy (for example by agitating) or heat energy. The incubation or release time may be shortened by increasing the quantity of energy put into the system.

Preferably, heat energy is put into the system by heating the nucleic acid to an elevated temperature for a predetermined time, while it is retained by the filter, prior to elution, but not so hot or for such a time as to be damaged. (However, elution still may be effected when the nucleic acid has not been heated to an elevated temperature or even has been held at a lowered temperature (as low as 4° C.) prior to elution.) More preferably, the nucleic acid is heated to an elevated temperature in the range of 40° C. to 125° C., even more preferably in the range of from 80° C. to 95° C. Most preferably, the nucleic acid is heated to an elevated temperature of about 90° C., advantageously for about 10 minutes for a filter having a 6 mm diameter. Increasing the filter diameter increases the yield of DNA at any given heating temperature. Heating may be required for genomic DNA, but for RNA and plasmid, heating is not necessary.

For DNA isolations, the ratio of double to single stranded DNA is dependent upon, and can be controlled by, the experimental conditions. Modifying the incubation regime using the parameters of time and temperature will alter this ratio, where a lower elution temperature over a longer time period will produce a high proportion of double stranded DNA. A higher elution temperature over a shorter period of time also will produce a higher proportion of double stranded DNA. Proteins may also be used to inhibit denaturation of DNA (see, e.g., WO 01/96351 (PCT/GB01/02564; filed 11 Jun. 2001) and WO 03/050278 (PCT/GB02/05617; filed 11 Dec. 2002)).

Once the nucleic acid has been heated to an elevated temperature while retained by the filter, it is not necessary to maintain the nucleic acid at the elevated temperature during elution. Elution itself may be at any temperature. For ease of processing, it is preferred that, where the nucleic acid is heated to an elevated temperature while retained by the filter, elution will be at a temperature lower than the elevated temperature. This is because when heating has been stopped, the temperature of the nucleic acid will fall over time and also will fall as a result of the application of any ambient temperature eluting solution to the filter. Alternatively, the process may be carried out in a heating element, such as in a heat block (e.g., in an automated device or in a robot).

Any solution at any pH which is suitable for eluting the nucleic acid from the present filter may work. Preferred elution solutions include NaOH 1 mM to 1 M, Na acetate 1 mM to 1M, 10 mM 2-[N-morpholino]-ethanesulfonic acid (MES) (pH 5.6), 10 mM 3-[cyclohexylamino]-1-propanesulfonic acid (CAPS) (pH 10.4), TE (10 mM Tris HCL (pH8)+1 mM EDTA), TE⁻¹ (10 mM Tris; 0.1 mM EDTA; pH 8), sodium dodecyl sulfate (SDS) (particularly 0.5% SDS), TWEEN™ 20 (particularly 1% TWEEN™ 20), LDS (particularly 1% lauryl dodecyl sulfate (LDS)) or TRITON™ or TRITON™-X-100 (particularly 1% TRITON™), water and 10 mM Tris. (TWEEN™ 20 is known by the names of sorbitan mono-9octadecenoate poly(oxy-1,1-ethanedlyl), polyoxyethylenesorbitan monolaurate, and polyoxyethylene (20) sorbitan monolaurate. The CAS number for the chemical is 9005-64-5. TRITON™-X-100 is known by the name of t-octylphenoxypolyethoxyethanol. The CAS number for t-octylphenoxypolyethoxyethanol is 9002-93-1.) For examples of elution protocols, see PCT/US01/25709 (filed 17 Aug. 2001), PCT/US02/36483 (filed 13 Nov. 2002), U.S. Pat. No. 6,645,717 (granted 11 Nov. 2003), and U.S. Pat. No. 6,670,128 (granted 30 Dec. 2003), the disclosures of all of which are incorporated herein by reference.

This device is not intended to be limited to the elution of DNA or limited to FTA® punches. The device is applicable to any material of interest deposited on any type of punch (e.g., paper or other cellulose-based matrices, glass and other silica-based matrices, plastic matrices, other membranes, etc.) that can be eluted from the punch. Examples of such materials include, but are not limited to, nucleic acid (e.g., genomic DNA, plasmid DNA, mitochrondrial DNA, cDNA, BAC, an oligonucleotide, viral DNA or RNA, mRNA, rRNA, tRNA, siRNA, and total RNA, etc.), protein(s) or any other material of interest (e.g., peptide, oligonucleotide, etc.).

In processing the FTA® punches, it has been observed that unlike genomic DNA, RNA does not remain on the FTA® paper during processing with 2×5 min washes with TE⁻¹ (10 mm Tris-HCl pH 8.0, and 0.1 mM EDTA) at room temperature. Virtually all of the RNA elutes into the initial wash, and this eluted cellular RNA can be directly placed into the first strand RT reaction or can be ethanol precipitated from the wash solution and resuspended in sterile water or TE prior to analysis. WO 01/501601 (PCT/US01/00640; filed 10 Jan. 2001), the disclosure of which is incorporated herein by reference. Materials and reagents must be RNAse-free for work with RNA (Sambrook et al., 1989).

Similar conditions may be used for elution of protein or peptide samples. For example, protein or peptide samples may be eluted with any appropriate protein buffer. In one embodiment, protein is eluted by incubation with phosphate buffered saline (PBS) (10×PBS: 137 mM NaCl; 2.7 mM KCl; 5.4 mM Na₂HPO₄; 1.8 mM KH₂PO₄; pH 7.4) for, e.g., 30-45 mins. WO 03/020924 (PCT/GB02/04048). Alternatively, high salt buffers (e.g., 0.1 M Tris-acetate with 2.0 M NaCl, (pH 7.7)), low salt buffers (e.g., 0.01 M Tris-HCl buffer (pH 8.0)), high pH buffers (e.g., 0.1 M Glycine-NaOH (pH 10.0), or low pH buffers (e.g., 0.1 M Glycine-HCl (pH 2.3)) may be used.

If the sample of interest is not eluted, it may be treated in situ, using the device of the present invention, for purposes of analysis, such as detection, as described in PCT/US02/36978 (filed 15 Nov. 2002), U.S. Pat. No. 6,746,841 (granted 8 Jun. 2004), and U.S. Ser. No. 10/298,255 (filed 15 Nov. 2002), the disclosures of all of which are incorporated herein by reference. For example, the detection process may comprise use of an indicator. The signal generated by the indicator of the present invention provides positive identification of the presence of a given nucleic acid or protein on the substrate. For example, nucleic acids can be detected (and preferably quantified) by the use of a specific or non-specific nucleic acids probe or other signal generators and one of the versions of immunoassay. Proteins can be detected (and preferably quantified) by the use of an immunoassay. Preferably, the indicator comprises a fluorescent indicator, a color indicator, or a photometric indicator. Alternatively, antibodies conjugated with biotin and polyavidin-horse radish peroxidase (HRP) may be used, or an assay using polyethyleneimine-peroxidase conjugate (PEI-PO), which interacts with DNA, may be used, as known in the art. Other methods of detection will occur to those of ordinary skill in the art.

In some embodiments, particularly in photosensitive embodiments, it may be necessary to provide a housing that inhibits exposure to light in general and/or to certain wavelengths of light in particular. If the indicator is not already present on the filter, it may be added and, if necessary, incubated with the filter material in the housing. The indicator is easily drawn through the filter material and discarded. Blocking agents and washes may likewise be circulated through the filter material, although in preferred embodiments, blocking is not necessary. When the preparation steps are complete, the housing is opened in the absence of light (or in the absence light of the wavelength for the indicator reaction), and the filter material is the exposed to the light of the desired wavelength to trigger the photometric reaction.

Other analytical methods may include hybridization of nucleic acids or proteins to the sample of interest, detection of the sample, quantification of the sample, identification or other testing of the sample, and other methods, which will occur to one of ordinary skill in the art.

In addition, it is important to maintain a record of the materials processed, the apparatus utilized and the processing results as the samples move through the various processing steps. In those cases in which punch processing is done by hand, manual record keeping and tracking typically is satisfactory. When processing is automated, however, means for tracking all of the materials processed (punches and their respective source cards), the apparatus elements utilized in the various processing steps for each sample, and the processing results for each sample become cumbersome but remain very important. As an example of a tracking means suitable for use with an automated processing capability, the present inventors have found that the use of bar code readers and bar code identifiers associated with at least (i) the cards from which the various punches are taken, (ii) the apparatus elements utilized in the processing of each punch utilized to process the respective punches, and (iii) the results of processing for each punch facilitate the tracking and record keeping functions. It is to be understood, however, that other automated tracking and record keeping means also may be utilized for the above purposes without departure from the present invention.

Advantages of the system include:
(a) Easy and secure handling of punches.
(b) Shallow design of punch support section allows for easy placement of punch.
(c) Large buffer volumes needed during washing stages as well as small buffer volumes needed during elution can be handled efficiently.
(d) Washing and elution can be handled by a standard liquid handling robot.
(e) Can be scaled to accommodate different punch sizes.
(f) Gentler on the punch. de-lamination of the punch during processing is eliminated and fiber shedding is minimized.
(g) Can be used for automated and manual handling.

### Definitions

The following additional definitions are provided for specific terms, which are used in the written description.

As used in the specification and claims, the singular form “a”, “an” and “the” include plural references unless the context clearly dictates otherwise. For example, the term “a molecule” also includes a plurality of molecules.

The term “filter membrane” or “matrix” as used herein means a porous material or filter media. As used herein, a “filtration medium,” “filter medium,” or “porous medium” may have uniform or non-uniform pores. Alternatively, it may comprise, for example, a “matrix of fibers” or a “network of fibers” through which appropriately smaller sized materials can pass. It may be loose material having an irregular composition, or it may have a more uniform or discrete composition. It includes, but is not limited to, a “filter,” a “filter membrane,” and a “matrix,” which, as used herein, mean a formed porous material or filter medium. It includes, but is not limited to, a “solid medium,” such as a “dry solid medium.” A “filtration medium,” “filter medium,” or “porous medium” may be formed, either fully or partly, from glass, silica, silica gel, silica oxide, or quartz, including their fibers or derivatives thereof, but is not limited to such materials. Other materials include, but are not limited to, nylon, cellulose-based materials (e.g., cellulose, nitrocellulose, carboxymethylcellulose, cellulose nitrate, cellulose acetate), hydrophilic polymers including synthetic hydrophilic polymers (e.g., polyester, polyamide, carbohydrate polymers), polytetrafluoroethylene, PET, polycarbonate, porous ceramics, as well as other materials disclosed herein. Filters based on metal oxides such as an aluminum oxide membrane (Whatman ANOPORE™) are also included. The filtration medium may comprise a filter or a plurality of filters.

As used herein, “hydrophilic” substance is one that absorbs or adsorbs water, while a “hydrophobic” substances is one that does not absorb or adsorb water.

As used herein, “wettable” refers to a membrane which is wetted across its entire surface without phobic patches.

The media used for the filter membrane of the invention includes any material that does not inhibit the storage, elution and subsequent analysis of sample material added to it. This includes flat dry matrices or a matrix combined with a binder. In one aspect, the support of the present invention allows for elution of the genetic material therefrom in a state that allows for subsequent analysis.

The medium can be combined with a “binder,” which holds the fibers together. Some examples of binders well-known in the art are polyvinylacrylamide, polyvinylacrylate, polyvinylalcohol (PVA), polystyrene (PS), polymethylmethacrylate (PMMA), and gelatin.

The term “integrity maintainer” or “integrity maintenance means” as used herein means a sealable member that prevents degradation and/or loss of the matrix. Preferably, the integrity maintainer of the present invention creates an air tight seal, thus preventing air, bacteria or other contaminants from coming into contact with the matrix and purified nucleic acid. The integrity maintainer can be in the form of a plastic bag, with or without a seal, cellophane, a sealable container, parafilm and the like.

As used herein, “storage” refers to maintaining the support/nucleic acids for a period of time at a temperature or temperatures of interest. Storage temperature and time depend on the type of membrane and the nature of the sample. For example, for DNA stored on a FTA® membrane, storage is preferably accomplished at about 20 to 30° C. (preferably room temperature, e.g. 25° C.), but may be at higher or lower temperatures depending on the need. Lower storage temperatures may range from about 0 to 20° C., −20 to 0° C., and −80 to −20° C. Long term storage in accordance with the invention is greater than one year, preferably greater than 2 years, still more preferably greater than 3 years, still more preferably greater than 5 years, still more preferably greater than 10 years, and most preferably greater than 15 years.

As used herein, an “analyte” is the element of the sample to be detected or isolated. In some embodiments, the analyte specifically binds a binding reagent. In some embodiments, the presence or absence of the analyte may be used to determine the physiological condition of an organism from which the sample was obtained. Alternatively, the presence or absence of the analyte may be used to detect, for example, contamination of a sample. A wide range of other uses will occur to one of skill in the art.

As used herein, “specificity” refers to the ability of an antibody to discriminate between antigenic determinants. It also refers to the precise determinants recognized by a particular receptor or antibody. It also refers to the ability of a receptor to discriminate between substrates, such as drugs. With respect to nucleic acids, it refers to identity or complementarity as a function of competition or recognition/binding, respectively. “Specificity” of recognition or binding may be affected by the conditions under which the recognition or binding takes place (e.g., pH, temperature, salt concentration, and other factors known in the art).

As used herein, a “ligand” is a molecule or molecular complex that can be bound by another molecule or molecular complex. The ligand may be, but is not limited to, a molecule or molecular complex bound by a receptor, or it may be a complementary fragment of nucleic acid.

As used herein, a “chimeric DNA” is at least two identifiable segments of DNA the segments being in an association not found in nature. Allelic variations or naturally occurring mutational events do not give rise to a chimeric DNA as defined herein.

“Nucleotide” as used herein refers to a base-sugar-phosphate combination. Nucleotides are monomeric units of a nucleic acid sequence (DNA and RNA). The term nucleotide includes ribonucleoside triphosphate ATP, UTP, CTG, GTP and deoxyribonucleoside triphosphates such as dATP, dCTP, dITP, dUTP, dGTP, dTTP, or derivatives thereof.

Such derivatives include, for example, [αS]dATP, 7-deaza-dGTP, 7-deaza-dATP, and biotinylated or haptenylated nucleotides. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels.

As used herein, the terms “polynucleotide” and “nucleic acid molecule” are used interchangeably to refer to polymeric forms of nucleotides of any length, which may have any three-dimensional structure, and may perform any function, known or unknown. The polynucleotides may contain deoxyribonucleotides (DNA), ribonucleotides (RNA), and/or their analogs, including, but not limited to, single-, double-stranded and triple helical molecules, a gene or gene fragment, exons, introns, messenger RNA (mRNA), transfer RNA (tRNA), ribosomal RNA (rRNA), small interfering RNA (siRNA), ribozymes, antisense molecules, complementary DNA (cDNA), genomic DNA (gDNA), recombinant polynucleotides, branched polynucleotides, aptamers, plasmids, vectors, isolated DNA of any sequence, isolated RNA of any sequence, nucleic acid probes, peptide nucleic acids (PNA), and primers. A nucleic acid molecule may also comprise modified nucleic acid molecules (e.g., comprising modified bases, sugars, and/or internucleotide linkers).

“Library” as used herein refers to a set of nucleic acid molecules (circular or linear) which is representative of all or a portion or significant portion of the DNA content of an organism (a “genomic library”), or a set of nucleic acid molecules representative of all or a portion or significant portion of the expressed genes (a “cDNA library”) in a cell, tissue, organ or organism. Such libraries may or may not be contained in one or more vectors.

“Vector” as used herein refers to a plasmid, cosmid, phagemid or phage DNA or other DNA molecule which is able to replicate autonomously in a host cell, and which is characterized by one or a small number of restriction endonuclease recognition sites at which such DNA sequences may be cut in a determinable fashion without loss of an essential biological function of the vector, and into which DNA may be inserted in order to bring about its replication and cloning. The vector may further contain one or more markers suitable for use in the identification of cells transformed with the vector. Markers, for example, include but are not limited to tetracycline resistance or ampicillin resistance. Such vectors may also contain one or more recombination sites, one or more termination sites, one or more origins of replication, and the like.

A “vector” is a replicon, such as plasmid, phage or cosmid, to which another DNA segment may be attached so as to bring about the replication of the attached segment. A “replicon” is any genetic element (e.g., plasmid, chromosome, virus) that functions as an autonomous unit of DNA replication in vivo, i.e., capable of replication under its own control.

Examples of “vectors” include plasmids, autonomously replicating sequences (ARS), centromeres, cosmids and phagemids. Vectors can further provide primer sites, e.g., for PCR, transcriptional and/or translational initiation and/or regulation sites, recombinational signals, replicons, etc. The vector can further contain one or more selectable markers suitable for use in the identification of cells transformed or transfected with the vector, such as kanamycin, tetracycline, amplicillin, etc.

“Primer” as used herein refers to a single-stranded oligonucleotide that is extended by covalent bonding of nucleotide monomers during amplification or polymerization of a DNA molecule. Preferred primers for use in the invention include oligo(dT) primers or derivatives or variants thereof.

“Oligonucleotide” as used herein refers to a synthetic or natural molecule comprising a covalently linked sequence of nucleotides which are joined by a phosphodiester bond between the 3′ position of the deoxyribose or ribose of one nucleotide and the 5′ position of the deoxyribose or ribose of the adjacent nucleotide.

“Template” as used herein refers to double-stranded or single-stranded nucleic acid molecules which are to be amplified, synthesized or sequenced. In the case of a double-stranded molecules, denaturation of its strands to form a first and a second strand is preferably performed before these molecules may be amplified, synthesized or sequenced, or the double stranded molecule may be used directly as a template. For single stranded templates, a primer, complementary to a portion of the template is hybridized or annealed under appropriate conditions and one or more polymerases or reverse transcriptases may then synthesize a nucleic acid molecule complementary to all or a portion of said template. The newly synthesized molecules, according to the invention, may be equal or shorter in length than the original template.

An “amino acid” refers to either natural and/or unnatural or synthetic amino acids, including glycine and both D or L optical isomers, and amino acid analogs and peptidomimetics. “Amino acids” also includes imino acids. A “peptide” is a compound of two or more subunit amino acids, amino acid analogs, or peptidomimetics. The subunits may be linked by peptide bonds or by other bonds (e.g., as esters, ethers, and the like). An “oligopeptide” refers to a short peptide chain of three or more amino acids. If the peptide chain is long (e.g., greater than about 10 amino acids), the peptide is a “polypeptide” or a “protein.”

While the term “protein” encompasses the term “polypeptide”, a “polypeptide” may be a less than full-length protein. In other respects, the terms “polypeptide” and “protein” are used interchangeably and refer to any polymer of amino acids (dipeptide or greater) linked through peptide bonds or modified peptide bonds. Thus, the terms “polypeptide” and “protein” include oligopeptides, protein fragments, fusion proteins and the like. It should be appreciated that the terms “polypeptide” and “protein”, as used herein, includes moieties such as lipoproteins and glycoproteins.

As used herein, a “chimeric protein” or “fusion protein” is a protein with at least two identifiable segments, the segments being in an association not found in nature. In one embodiment, a chimeric protein may arise, for example, from expression of a chimeric DNA capable of being expressed as a protein and having at least two segments of DNA operably linked to enable expression of at least a portion of each segment as a single protein. Other embodiments will suggest themselves to one of ordinary skill in the pertinent art.

A “prion” is a protein or protein fragment capable of replicating.

A “tag peptide sequence” is a short peptide or polypeptide chain of 3 or more amino acids, which is attached to a protein of interest. In a preferred embodiment, a polypeptide, protein, or chimeric protein comprises a tag peptide sequence, which is used for purification, detection, or some other function, such as by specific binding to an antibody. The antibody may be in solution or bound to a surface (e.g., a bead, filter, or other material). The tag peptide sequence should not interfere with the function of the rest of the polypeptide, protein, or chimeric protein. An example of a tag peptide sequence useful in the present invention is a short c-Myc tag with six His residues fused at the carboxyl-terminus. Other examples will be well-known to those of ordinary skill in the pertinent art.

“Conservatively modified variants” of domain sequences also can be provided within the scope of the invention. With respect to particular nucleic acid sequences, conservatively modified variants refers to those nucleic acids which encode identical or essentially identical amino acid sequences, or where the nucleic acid does not encode an amino acid sequence, to essentially identical sequences. Specifically, degenerate codon substitutions can be achieved by generating sequences in which the third position of one or more selected (or all) codons is substituted with mixed-base and/or deoxyinosine residues. Alternatively, one or more amino acids may be substituted with an amino acid having a similar structure, activity, charge, or other property. Conservative substitution tables providing functionally similar amino acids are well-known in the art (see, e.g., Proc. Natl. Acad. Sci. USA 89:10915-10919 (1992)).

The source of the nucleic acid or protein can be a biological sample containing whole cells. The whole cells can be, but are not restricted to, blood, bacterial culture, bacterial colonies, yeast cells, tissue culture cells, saliva, urine, drinking water, plasma, stool samples, semen, vaginal samples, sputum, plant cell samples, or various other sources of cells known in the scientific, medical, forensic, and other arts. The samples can be collected by various means known in the art, transported to the filter, and then applied thereto.

A “host organism” is an organism or living entity, which may be prokaryotic or eukaryotic, unicellular or multicellular, and which is desired to be, or has been, a recipient of exogenous nucleic acid molecules, polynucleotides, and/or proteins. Preferably, the “host organism” is a bacterium, a yeast, or a eukaroytic multicellular living entity (preferably an animal, more preferably a mammal, still more preferably a human).

An “antibody” (Ab) is protein that binds specifically to a particular substance, known as an “antigen” (Ag) (described infra). An “antibody” is any immunoglobulin, including antibodies and fragments thereof, that binds a specific epitope. The term encompasses polyclonal, monoclonal, and chimeric antibodies (e.g., multispecific antibodies).

An “antigen” (Ag) is any substance that reacts specifically with antibodies or T lymphocytes (T cells). An “antigen-binding site” is the part of an immunoglobulin molecule that specifically binds an antigen.

“Biological sample” includes samples of tissues, cells, blood, fluid, or other materials obtained from a biological organism. It also includes a biological organism, cell, virus, or other replicative entity. Also included are solid cultures (such as bacterial or tissue cultures). Also included are solid samples, including, but not limited to, food, powder, and other solids, including non-biological solids, containing a biological organism, cell, virus, or other replicative entity. Also included are washing, homogenizations, sonications, and similar treatments of solid samples. Likewise, the term includes non-solid biological samples.

A “weak base” has an alkaline pH between 8.0 and 9.5 or causes an alkaline pH between 8.0 and 9.5.

As used herein, “non-ionic interactions” include any interactions in the absence of ionic interaction. “Non-ionic interactions include, but are not limited to, dipole-dipole interactions, dipole-induced dipole interactions, dispersion forces, or hydrogen bonding,

When not otherwise stated, “substantially” means “being largely, but not wholly, that which is specified.”

Various aspects and embodiments of the present invention will now be described in more detail by way of example. It will be appreciated that modification of detail may be made without departing from the scope of the invention.

### EXAMPLES

### Examples 1-3

The device consists essentially of two elements or sections (10, 100), shown supported by holders (60, 160) in FIGS. 1-3. The first element (10), here the lower section, is shown in FIG. 1. The first element (10) is depicted as a dispensing pipet tip having a hollow internal shaft (22) with an external opening (24) and a first housing assembly (12). The first housing assembly (12) houses the first inner reservoir (14).

The punch (20) (such as a punch from an FTA® CLONESAVER® archival card) is placed in this section of the device by punch and place equipment. In this embodiment, the punch rests on a punch support (50). Once the punch (20) is placed in the first inner reservoir (14), it divides the first inner reservoir (14) into a first chamber (16), which communicates with the hollow internal shaft (22), and a second chamber (18), which has a coupling opening (32). The coupling opening (32) is defined by an edge or rim (30).

In the embodiment depicted in FIG. 1, the distance between the top edge (30) of the lower section (10) and the punch support (50) is kept shallow to allow easy placement of the punch (20). In this embodiment, the tip (40) of the lower section (10) is shown as a capillary tip, such as a gel loading tip, which minimizes the hold up volume of the tip.

The second element, or upper section (100) of this embodiment of the device is depicted in FIG. 2. In FIG. 2, the upper section (100) is depicted as a dispensing pipet tip, which has a second housing assembly (110) defining a hollow interior (120).

In this embodiment, this second dispensing pipet tip is larger than the first tip used as the lower section (10). The second housing assembly (110) is structured and arranged to define a coupling portion (130) for communication through the first coupling opening (32) of the lower section (10), between the hollow interior (120) of the upper section (100) and the second chamber (18) of the first inner reservoir (14). In this embodiment, the coupling portion (130) ends with a rim or edge (150), which defines the second coupling opening (140), which communicates with the lower section (10) (see FIGS. 2 and 3). This part is picked up directly or indirectly by a user or by a standard liquid handling robot and pushed into the lower section in a manner similar to that shown in FIG. 3.

FIG. 3 depicts the two elements, or sections, joined. As shown in FIG. 2, the dimensions of the second housing assembly (110) are selected such that, when a punch (20) is positioned in the first inner reservoir (14) of the lower section (10), the rim or edge (150) defining the second coupling opening (140) contributes to maintain the position of the punch (20) within the first inner reservoir (14) and forms a second inner reservoir (200) housed within the hollow interior (120) of the second housing assembly (110), which is interior to the second chamber (18) of the lower section (10). When attached to a pipetter (220), force is communicated from the pipettor (220) through an opening (170) in the upper section (100) into the hollow interior (120) through the second inner reservoir (200). Once the assembly is locked in place, the pipettor is used to move the assembly out of the holder and into the buffer stations and so forth.

Once the two parts are pushed together, the two sections lock together (e.g., by pressure or by a locking mechanism or element), and the device is moved as a single piece. Once the upper and lower sections are locked together, the user or liquid handling robot picks up the device and takes the device to a buffer station, as depicted in FIG. 4. The sample on the punch (20) is washed by aspirating liquid (e.g., hot or cold aqueous buffers, alcohols, organic solvents, etc.) into the device and pulsing the liquid up and down through the punch or by aspirating and dispensing liquid from the device.

After washing, the elution buffer is aspirated in to the device and incubated. Buffer is taken up in to the tip and moved up and down through the punch to wash and elute. If heating is necessary at the elution stage the whole tip is placed in a heating block. After incubation, the liquid from the device is dispensed in to a collection vessel, and the device is discarded. The design makes possible the use of relatively large (e.g., 200 μl) buffer volumes during washing stages and the use of small volumes during the elution stage.

In Example 1, the above device is used in a single-channel pipettor. In Example 2, the device is used in a multi-channel pipettor. In Example 3, the device is used in a robotic pipettor as described above.

### Example 4-6

An alternative to the user punching the sample matrix and placing the punch in the device (e.g., the device of Example 1 or Example 2) is for the manufacturer of the device to provide a device pre-loaded with a punch (e.g., providing a lower section pre-loaded with a FTA® punch). The user then pushes the upper section (Example 4) or sections (Example 5) into place (i.e., assembling the upper and lower sections together). The spot is allowed to dry and the devices are then placed in storage until required. When needed, the device (Example 4) or devices (Example 5) are processed as described above to obtain the material of interest (e.g., DNA, RNA, protein, etc.).

Similarly, an alternative to the user punching the sample matrix and placing the punch in the device (e.g., the device of Example 3) is for the manufacturer of the device to provide a device pre-loaded with a punch (e.g., providing a lower section pre-loaded with a FTA® punch). In Example 6, the user then loads the lower sections into liquid handling robot and spots the sample of interest onto the punch using the robot.

The upper sections are then pushed into place (i.e., assembling the upper and lower sections together). The spot is allowed to dry and the devices are then placed in storage until required. When needed, the devices are processed as described above to obtain the material of interest (e.g., DNA, RNA, protein, etc.).

### Examples 7 and 8

As an alternative to the device of Example 1, a modified syringe device may be used (e.g., as in FIG. 5A or FIG. 5B), fitted either to a first element similar to the lower section described above or to a modified needle. The size of the needle is selected to prevent loss of the punch. Alternatively, the size of the punch is selected to prevent its loss when using a specifically sized needle. In addition, the length and bore of the needle are selected with reference to the sample of interest being isolated. For example, a wide bore needle is used when the sample of interest comprises long strands of DNA, because shear forces in a narrow bore needle can break the strands.

In FIG. 5A, a first element (lower section), analogous to that of FIGS. 1-4, is shown attached to a syringe (300), the housing assembly (310) of which is structure and arranged to define the hollow interior (320) of the syringe (300). A coupling portion (330) of the syringe (300) couples the syringe (300) to the coupling opening of the first element so that the opening (340) of the syringe (300) communicates with the first inner reservoir. The rim or edge (350) of the opening contributes to maintaining the position of the punch (440), which is positioned on a punch support (450). The two sections lock together (e.g., by pressure or by a locking mechanism or element, such as a snap fitting or a Luer lock). The syringe plunger (380) is used to draw liquid in and out.

In FIG. 5B, the first element (lower section) (400) has a housing assembly (410) connected to a syringe or other needle (420). The rim or edge (350) of the opening contributes to maintaining the position of the punch (460), which is positioned on a punch support (470).

In Example 7, the syringe needle is modified to have an extra-long connector region, which connects the upper end of the needle to the bottom end of the syringe. The connector region has a punch support onto which the punch is placed prior to attachment to the syringe. Alternatively, the connector region decreases in interior radius so that the punch rests on the interior wall, but with a space below it to maintain room for buffers or other liquids between the underside of the punch and the top end of the needle. The upper side of the punch is pressed down by the nozzle of the syringe when the syringe is attached. Alternatively, an O-ring is inserted prior to attachment to the syringe.

In Example 8, an adaptor is provided and is placed between the syringe needle and the syringe. The adaptor is capable of fastening to the syringe nozzle at its upper end and to the connector region of the needle at its lower end, preferably using standard methods to allow commercially available syringes and needles to be used. The adaptor optionally has a punch support onto which the punch is placed prior to attachment to the syringe. The upper side of the punch is pressed down by the nozzle of the syringe when the syringe is attached. Alternatively, an O-ring is inserted prior to attachment to the syringe.

### Example 9

As an alternative to the device of Example 1, a modified syringe device may be used. In this embodiment, a lower section is provided as described in Example 1, but it is modified so that it can be attached to a syringe or the syringe is modified so that it can be attached to the lower section. The upper side of the punch is pressed down by the nozzle of the syringe when the syringe is attached. Alternatively, an O-ring is inserted prior to attachment to the syringe.

### Examples 10-12

Examples 10-12 are similar to Example 4, but using the devices of Examples 7-9.

In Example 10, as an alternative to Example 7, the manufacturer of the device of Example 7 provides a device pre-loaded with a punch (e.g., provides a modified syringe needle with an extra-long connector pre-loaded with a FTA® punch). The user then spots the sample of interest onto the punch and attaches the syringe. The spot is allowed to dry, and the device is then placed in storage until required. When needed, the device is processed as described above to obtain the material of interest (e.g., DNA, RNA, protein, etc.).

In Example 11, as an alternative to Example 8, the manufacturer of the device of Example 8 provides a device pre-loaded with a punch (e.g., provides a modified syringe needle with an extra-long connector pre-loaded with a FTA® punch). The user then spots the sample of interest onto the punch and attaches the syringe. The spot is allowed to dry, and the device is then placed in storage until required. When needed, the device is processed as described above to obtain the material of interest (e.g., DNA, RNA, protein, etc.).

In Example 12, as an alternative to Example 9, the manufacturer of the device of Example 9 provides a device pre-loaded with a punch (e.g., provides a lower section pre-loaded with a FTA® punch). The user then spots the sample of interest onto the punch and attaches the syringe. The spot is allowed to dry, and the device is then placed in storage until required. When needed, the device is processed as described above to obtain the material of interest (e.g., DNA, RNA, protein, etc.).

### Example 13

The device of Example 1 is used to isolate RNA from a punch. In processing RNA sample punches, however, the materials and solutions used are RNAse-free, according to methods known in the art (see, e.g., Sambrook et al., Molecular Cloning: A Laboratory Manual (2d ed.), Cold Spring Harbor Laboratory Press, Cold Spring Harbor, N.Y. (1989)).

### Example 14

The device of Example 1 is used to process a protein punch. In this example, protein is eluted by incubation with phosphate buffered saline (PBS) (PBS: 137 mM NaCl; 2.7 mM KCl; 6.3 mM Na₂HPO₄; 1.47 mM KH₂PO₄; pH 7.4) for, e.g., 30-45 mins.

### Example 15

An experiment was carried out to show that plasmid DNA can be eluted from an indicating FTA® (Whatman) punch using a device and method similar to the device and method described above. A device was constructed using a 200 μl pipet as the lower part of the device and a 1000 μl pipet tip as the upper part of the device.

A 7 mm FTA® punch previously spotted with plasmid DNA (pGEM-luc) was inserted into a 200 μl pipet tip. A 1000 μl pipet tip was inserted into the 200 μl tip until it rested on top of the punch. (The 1000 μl tip had been shortened at the tip end to yield the length required to hold the punch in position within the 200 μl tip.)

The plasmid DNA was eluted from the punch as follows:
a) The two-tip assembly was attached to a 1000 μl pipettor;
b) 100 μl of TE⁻¹ (10 mM Tris HCl (pH 8) with 100 μM EDTA (ethylene diamine tetra-acetic acid)) was taken into the tip, and the liquid was allowed to contact the punch;
c) After a period of incubation (at room temp) with the TE-1, the liquid was moved up and down past the punch three times by use of the pipettor; and
d) The liquid was then collected in a microcentrifuge tube. An aliquot (2 μl) of this eluate was used in the transformation reaction.

The transformation by electroporation was carried out as follows:
1) 2 μl of eluate containing plasmid DNA (or pUC19 DNA as a standard or TE⁻¹ as a negative control) was added to a chilled microcentrifuge tube (1.8 ml tube).
2) 20 μl of competent cells (ElectroMAX-DH5^{α}, Invitrogen) was added to the above tube.
3) The sample was mixed very gently.
4) The resulting mixture was incubated 10 min.on ice.
5) The full amount of the mixture was transferred to a chilled electroporation cuvette (0.1 cm gap).
6) The cuvette was placed in a Bio-Rad Micropulser and a pulse (prog. Ec1) was applied. The Ec1 program applies a voltage of 1.8 kV when a 0.1 cm gap cuvette is used.
7) Immediately, 980 μl of S.O.C. medium (2% bacto-tryptone, 0.5% yeast extract, 0.05% NaCl, 2.5 mM KCl, 10 mM MgCl₂ (pH 7.0) (with 20 mM glucose)) was added.
8) The liquid from the cuvette was transferred to a round 14 ml Falcon bottom tube (BD product code 2059).
9) The sample was incubated for 1 hr at 37° C. with mixing at 225 rpm.
10) Samples were diluted in S.O.C. medium to 1:20 and the pUC standard was diluted to 1:100.
11) 100 μl of each dilution was spread onto a LB/Ampicillin plate (Luria Broth (LB): 1% bacto-tryptone, 0.5% yeast extract, 0.5% NaCl (pH 7.0) (with 100 μg/ml ampicillin (amp) and 12.5 g/L bacto-agar). Two plates were prepared for each diluted sample.

12) Plates were incubated at 37° C. overnight, and the colonies were counted (see Table 1 for results).

TABLE 1
Transformation using Electroporation

| | | Colony | Colony | Mean | | Transformation |
|---|---|---|---|---|---|---|
| | | Count | Count | Colony | | Efficiency |
| Sample # | Description | Plate A | Plate B | Count | Transformations | (CFU/μg) |
| 1 | FTA ® punch | 4656 | 4896 | 4776 | 955,200 | n/a |
| | (5 min. | | | | | |
| | Incubation) | | | | | |
| 2 | FTA ® punch | 2920 | 2848 | 2884 | 576,800 | n/a |
| | (10 min. | | | | | |
| | Incubation) | | | | | |
| 3 | FTA ® punch | 3760 | 2992 | 3376 | 675,200 | n/a |
| | (10 min. | | | | | |
| | Incubation) | | | | | |
| 4 | Blank | 0 | 0 | 0 | 0 | n/a |
| 5 | pUC control | 288 | 258 | 273 | 273,000 | 1.37E+10 |

The above results show that plasmid DNA can be eluted from an FTA® punch using the principle employed in the above-described device.

### REFERENCES

Sambrook et al., Molecular Cloning: A Laboratory Manual (2d ed.), Cold Spring Harbor Laboratory Press, Cold Spring Harbor, N.Y. (1989).

Throughout this application, various publications including United States patents, are referenced by author and year and patents by number. The disclosures of these publications and patents in their entireties are hereby incorporated by reference into this application in order to describe more fully the state of the art to which this invention pertains.

The invention has been described in an illustrative manner, and it is to be understood that the terminology which has been used is intended to be in the nature of words or description, rather than of limitation.

Obviously, many modifications and variations of the present invention are possible in light of the above teachings. It is, therefore, to be understood that within the scope of the described invention, the invention may be practiced otherwise than as specifically described.

What is claimed is:

1. A device for processing a punch from a matrix comprising a sample of interest, wherein the device comprises:
a. a first element comprising a dispensing tip comprising:
i. a hollow internal shaft having an external opening; and
ii. a first housing assembly structured and arranged to define a first inner reservoir communicating with the internal shaft of the dispensing tip, wherein the dimensions of the first inner reservoir are selected such that a punch inserted therein divides the first inner reservoir into two chambers, wherein:
the first chamber communicates with the internal shaft of the dispensing tip; and
the second chamber comprises a coupling opening; and
b. a second element comprising a second housing assembly structured and arranged to define:
i. a hollow interior;
ii. a coupling portion for engaging the second element to the first element; and
iii. a second coupling opening for communication through the first coupling opening of the first element, between the hollow interior of the second element and the first inner reservoir, wherein the dimensions of the second housing assembly are selected such that, when a punch is positioned in the first inner reservoir of the first element, the rim defining the second coupling opening contributes to maintain the position of the punch within the first inner reservoir and forms a second inner reservoir within the hollow interior of the second element.

2. The device of claim 1, wherein the first inner reservoir further comprises a punch support, wherein the dimensions of the punch support are selected such that most of the surface area of the punch placed on the punch support is accessible to a fluid in the first chamber when a punch is placed on the punch support and the first chamber is filled with the fluid.

3. The device of claim 2, wherein the punch support comprises an O-ring.

4. The device of claim 2, wherein the punch support comprises a ribbed support with a series of channels for fluid flow.

5. The device of any one of the preceding claims, wherein the coupling portion engages the second element to the first element by a pressure lock.

6. The device of any one of the preceding claims, wherein the coupling portion engages the second element to the first element by mechanical locking mechanism.

7. The device of claim 6, wherein the mechanical locking mechanism comprises a snap fitting or a Luer lock.

8. The device of any one of the preceding claims, wherein the first inner reservoir is contiguous with the hollow inner shaft within the first housing assembly.

9. The device of any one of the preceding claims, wherein the dispensing tip of the first element comprises a micro-dispensing pipet tip.

10. The device of claim 9, wherein the micro-dispensing pipet tip comprises a capillary micro-dispensing pipet tip.

11. The device of any one of the preceding claims, wherein the second element comprises a micro-dispensing pipet tip.

12. The device of any one of the preceding claims, wherein the device is adapted for use with a micro-dispensing pipet selected from the group consisting of:
d. a single-channel micro-dispensing pipet;
e. a multi-channel micro-dispensing pipet; and
f. an automated micro-dispensing pipet machine or robot.

13. The device of claim 1, wherein the second element comprises a syringe selected from the group consisting of:
a. a manual syringe; and
b. a robotic syringe.

14. The device of claim 13, wherein the first element is adapted to be fitted onto a syringe, wherein the hollow internal shaft is housed in a syringe needle.

15. The device of claim 14, wherein the first housing assembly is adapted to be fitted between a syringe and a syringe needle.

16. The device of any one of the preceding claims, further comprising:
c. a heating element.

17. A kit for processing a punch from a matrix comprising a sample of interest, wherein the kit comprises:
b. the device of any one of the preceding claims; and
b. a processing reagent.

18. The kit of claim 17, wherein the processing reagent comprises an elution buffer.

19. The kit of claim 18, wherein the elution buffer is selected from the group consisting of NaOH, sodium acetate, 10 mM 2-[N-morpholino]-ethanesulfonic acid (MES), 10 mM 3-[cyclohexylamino]-1-propanesulfonic acid (CAPS), TE, TE⁻¹, sodium dodecyl sulfate (SDS), an aqueous solution of sorbitan mono-9octadecenoate poly(oxy-1,1-ethanedlyl), lauryl dodecyl sulfate (LDS), or t-octylphenoxypolyethoxyethanol, 10 mM Tris, phosphate buffered saline (PBS), and water.

20. The kit of claim 17, wherein the processing reagent comprises an indicator.

21. The kit of claim 17, wherein the processing reagent comprises an enzyme or a photolytic agent.

22. The kit of claim 17, further comprising:
c. a heating element.

23. The kit of claim 17, further comprising:
c. a punch comprising a sample of interest.

24. The kit of claim 23, wherein the sample of interest comprises:
iv. a nucleic acid; or
v. a protein or a peptide.

25. The kit of claim 24, wherein the sample of interest comprises a nucleic acid selected from the group consisting of genomic DNA, plasmid DNA, mitochrondrial DNA, cDNA, an oligonucleotide, viral DNA or RNA, BAC, mRNA, rRNA, tRNA, siRNA, and total RNA.

26. A method of processing a punch from a matrix comprising a sample of interest, wherein the method comprises:
vi. punching a matrix to yield a punch comprising a sample of interest;
vii. providing the device of any one of the preceding claims;
viii. inserting the matrix into the first housing assembly of the first element of the device;
ix. coupling the first element to the second element;
x. processing the punch with a processing reagent.

27. The method of claim 26, wherein the processing step comprises:
xi. drawing the processing reagent into the first element so that the processing reagent contacts the punch;
xii. removing the processing reagent from the first element.

28. The method of claim 26, wherein the processing step comprises:
a. drawing the processing reagent into the first element so that the processing reagent contacts the punch and moves past it into the second chamber;
b. pushing the processing reagent through the punch into the first chamber;
c. removing the processing reagent from the first element.

29. The method of claim 27 or claim 28, wherein step i. further comprises incubation of the punch.

30. The method of any one of claims 26-28, wherein the coupling step comprises locking by pressure or by a mechanical lock.

31. The method of any one of claims 26-28, wherein the processing reagent comprises an elution buffer.

32. The method of claim 31, wherein the elution buffer is selected from the group consisting of NaOH, sodium acetate, 10 mM 2-[N-morpholino]-ethanesulfonic acid (MES), 10 mM 3-[cyclohexylamino]-1-propanesulfonic acid (CAPS), TE, TE⁻¹, sodium dodecyl sulfate (SDS), an aqueous solution of sorbitan mono-9octadecenoate poly(oxy-1,1-ethanedlyl), lauryl dodecyl sulfate (LDS), or t-octylphenoxypolyethoxyethanol, 10 mM Tris, phosphate buffered saline (PBS), and water.

33. The method of claim 31, wherein the elution buffer is heated to a temperature of between 40° C. to 125° C., wherein:
a. the elution buffer is heated prior to contact with the punch; or
b. the elution buffer is heated during contact with the punch in the first housing assembly.

34. The method of claim 31, wherein the elution buffer is heated to a temperature of between 65° C. and 95° C.

35. The method of any one of claims 26-28, wherein the processing reagent comprises an indicator.

36. The method of any one of claims 26-28, wherein the processing reagent comprises an enzyme or a photolytic agent.

37. The method of any one of claims 26-28, wherein the sample of interest comprises a nucleic acid.

38. The method of claim 37, wherein the nucleic acid is selected from the group consisting of genomic DNA, plasmid DNA, mitochrondrial DNA, cDNA, an oligonucleotide, BAC, viral DNA or RNA, BAC, mRNA, rRNA, tRNA, siRNA, and total RNA.

39. The method of any one of claims 26-28, wherein the sample of interest comprises a protein or a peptide.

40. The method of any one of claims 26-28, wherein the matrix comprises:
a. a cellulose-based matrix;
b. a silica-based matrix; or
xiii. a plastics-based matrix.
